(19) United States
(12) Patent Application Publication
(10) Pub. No.: US 2004/0259270 A1
(43) Pub. Date: Dec. 23, 2004

(54) System, device and method for exciting a sensor and detecting analyte

(76) Inventor: David E. Wolf, Sudbury, MA (US)

Correspondence Address: Allison Johnson, Allison Johnson, P.W., 6016 Logan Ave. S., Minneapolis, MN, 55419, US

(21) Appl. No.: 10/698,289

(22) Filed: Oct. 31, 2003

Related U.S. Application Data
(60) Provisional application No. 60/479,949, filed on Jun. 19, 2003.

(51) Int. Cl. G01N 33/543

(52) U.S. Cl. 436/518

(57) ABSTRACT

A system for detecting analyte that includes a sensor adapted to detect the analyte, the sensor including a polymer matrix, fluorophores and a membrane, an excitation source to excite a fluorophore of the sensor, a first detector adapted to detect light of a first wavelength emitted by the sensor, a second detector adapted to detect light of a second wavelength emitted by the sensor, and a processor for processing signals corresponding to light detected by the detectors.

### CROSS-REFERENCE TO RELATED APPLICATIONS

This application claims priority to U.S. Provisional Patent Application Ser. No. 60/479,949 filed on Jun. 19, 2003, entitled, “Device for Illuminating and Detecting Sensed Analyte.”

### BACKGROUND

The invention relates to exciting a sensor and detecting analyte.

It is often necessary to monitor an analyte in a body fluid to determine whether the analyte is present in the fluid and to determine the level of analyte in the fluid. Diabetes, for example, is a disease that requires close monitoring of glucose levels and administration of insulin the amount of which is dependent upon the glucose level. Ideally glucose levels are monitored on a continuous basis and insulin administration is continuously adjusted in response to any change in the blood glucose level.

Current methods for monitoring blood glucose levels in an individual include taking blood samples and monitoring urine glucose levels. Blood sampling is an invasive method and typically is not performed on a continuous basis. Urine glucose levels are not a direct measurement of blood glucose levels and are not necessary finely-tuned to the glucose level in blood.

It is desirable to provide noninvasive means for closely monitoring blood analyte levels, such as blood glucose levels, providing information related to such levels to the individual, and coordinating the delivery of medication, such as insulin dosages, with such levels. Various attempts have been made to develop such noninvasive methods. One area of research involves the development of implantable sensors for detecting the presence of analytes, such as glucose, in the body fluid of an individual. These sensors use a variety of mechanisms for detecting glucose including mechanisms that are based on fluorescence or fluorescence resonance energy transfer.

### SUMMARY

In one aspect, the invention features a system for detecting an analyte, the system including a sensor adapted to detect the analyte, the sensor including a polymer matrix, a fluorophore and a membrane surrounding the matrix, an excitation source to excite the fluorophore, a first detector adapted to detect light of a first wavelength emitted by the sensor, a second detector adapted to detect light of a second wavelength emitted by the sensor, and a processor for processing signals from the first and second detectors corresponding to light detected at the first and second detectors. In one embodiment, the excitation source is adapted to transcutaneously excite the fluorophore. In other embodiments, the detector is adapted to transcutaneously detect light emitted by the sensor.

In some embodiments, the system further includes a telemetry system for transmitting a signal corresponding to light detected at the first and second detectors to a remote location. In other embodiments, the system further includes a first filter to filter light received by the first detector. In another embodiment, the system further includes a second filter to filter light received by the second detector.

In one embodiment, the system further includes a first dichromatic mirror positioned to reflect light emitted by the excitation source and to transmit light emitted by the sensor. In another embodiment, the system further includes a second dichromatic mirror positioned to reflect a first wavelength of light emitted by the sensor and to transmit a second wavelength of light emitted by the sensor.

In other embodiments, the system further includes a fiber optic operatively connected to the excitation source. In one embodiment, the fiber optic includes a single mode optical fiber. In some embodiments, the system further includes a fiber optic operatively coupled to at least one of the detectors and adapted to transmit light from the sensor to the at least one detector.

In one embodiment, the system further includes a pump adapted to receive an instruction from the processor and to deliver an amount of a medicament, in response to the instruction.

In other embodiments, the fluorophores are mobile within the matrix.

In some embodiments, the processor is adapted to store a value corresponding to a property of the detected analyte as a function of time. In other embodiments, the processor is adapted to transfer a value corresponding to a property of the detected analyte to a remote device. In another embodiment, the processor is adapted to relate signals corresponding to the light detected by the detectors to a property of the analyte. In some embodiments, the property includes the concentration of the analyte. In another embodiment, the processor is adapted to provide instructions regarding an activity related to a property of the detected analyte. In other embodiments, the processor is adapted to provide instructions related to a property of the detected analyte to at least one of a mammal and a device. In another embodiment, the instructions include instructions to administer at least one of insulin, glucose or a combination thereof. In some embodiments, the processor is adapted to provide an alarm when a predetermined condition related to a property of the analyte is met.

In another embodiment, the sensor detects analyte selected from the group consisting of carbohydrates, glycoproteins, glycopeptides, enzymes, glycolipids, hormones, lipoproteins, antibodies, antigens, haptens, steroids, theophylline, creatinine, drugs, polynucleotides, pesticides, and combinations thereof. In one embodiment, the sensor detects glucose.

In one embodiment, at least one of the excitation source, the first detector and the second detector are located on at least one semiconductor wafer. In other embodiments, the detectors are adapted to simultaneously detect light received from the sensor.

In some embodiments, the system further includes a means for pulsing the light emitted by the excitation source. In another embodiment, the system further includes a means for phase locking the counting of signals at the detectors with the pulse emitted by the light pulsing means. In other embodiments, the system further includes a pump adapted to draw fluid from an individual for contact with the sensor. In one embodiment, the fluid includes interstitial fluid or blood.

In one embodiment the system for detecting an analyte includes a sensor adapted to detect the analyte, the sensor including a matrix, fluorophores, and a membrane, an excitation source to excite a fluorophore of the sensor, a first detector adapted to detect light of a first wavelength emitted by the sensor, a second detector adapted to detect light of a second wavelength emitted by the sensor, a third detector adapted to detect light of a third wavelength, and a processor for processing signals corresponding to light detected by the detectors and for determining a property of the analyte. In some embodiments, the excitation source is adapted to transcutaneously excite a fluorophore in the sensor. In other embodiments, the detectors are adapted to transcutaneously detect light emitted by the sensor.

In some embodiments, the system further includes a transmitter for transmitting signals corresponding to light detected by the detectors to a remote location. In other embodiments, the system further includes at least one filter to filter light received by at least one of the detectors. In other embodiments, the system further includes a dichromatic mirror positioned to reflect light emitted by the excitation source and to transmit light emitted by the sensor.

In another embodiment, the system further includes a second dichromatic mirror positioned to reflect a first portion of the light transmitted through the first dichromatic mirror and to transmit a second portion of the light transmitted through the first dichromatic mirror. In some embodiments, the system further includes a third dichromatic mirror positioned to reflect a first portion of the light transmitted through the second dichromatic mirror and to transmit a second portion of the light transmitted through the second dichromatic mirror.

In some embodiments, the system further includes a fiber optic operatively coupled to the excitation source. In other embodiments, the system further includes a fiber optic operatively coupled to at least one of the detectors to transmit light from the sensor to at least one detector. In another embodiment, the fiber optic includes a bundle of optical fibers, a first portion of the fibers being operatively connected to the first detector, a second portion of the fibers being operatively connected to the second detector, and a third portion of the fibers being operatively connected to the third detector.

In one embodiment, the third detector is adapted to detect light emitted by skin when the skin is excited by the excitation source.

In one embodiment, the processor is programmed with code to correct for the light emitted and scattered by the skin. In another embodiment, the processor is programmed with code to receive data corresponding to a first I(λ₁), a second I(λ₂), and a third I(λ_{B}) intensity measured at a first (λ₁), a second (λ₂), and a third (λ_{B}) wavelength, the third wavelength (λ_{B}) being selected such that the intensity detected at the third wavelength (λ_{B}) consists of background signal, correct the intensity at the first wavelength I(λ₁) based on the third intensity I(λ_{B}) and a first predetermined correction function B(λ₁), and correct the intensity at the second wavelength I(λ₂) based on the third intensity I(λ_{B}) and a second predetermined correction function B(λ₂).

In some embodiments, the processor further includes code to calculate a ratio of the corrected intensity at the first wavelength (λ₁) to the corrected intensity at the second wavelength (λ₂). In one embodiment, the processor further includes code to determine a property of the analyte. In some embodiments, the property is concentration.

In another embodiment, the processor is programmed with code to receive data corresponding to a first I(λ₁), a second I(λ₂), and a third I(λ_{B}) intensity at a first λ₁, a second λ₂, and a third λ₃ wavelength, respectively, correct the measured intensity at the first wavelength I(λ₁) based on the intensity at the third wavelength I(λ₃) and a first set of three predetermined correction functions D(λ₁), A(λ₁), B(λ₁), and correct the measured intensity at the second wavelength I(λ₂) based on the intensity at the third wavelength I(λ₃) and a second set of three predetermined correction functions D(λ₂), A(λ₂), B(λ₂).

In another aspect, the invention features a method of determining the concentration of an analyte using a system described herein, the method including exciting a fluorophore located in the sensor, detecting light of a first wavelength emitted by the sensor, detecting light of a second wavelength emitted by the sensor, and determining the concentration of the analyte based upon a corrected intensity of the light of the first wavelength and a corrected intensity of the light of the second wavelength. In some embodiments, the system further includes determining the ratio of the intensity of the light emitted by the sensor at the first wavelength to the intensity of the light emitted by the sensor at the second wavelength. In other embodiments, the system further includes determining the excited state fluorescence lifetime of the light emitted by the sensor at at least one of the first wavelength and the second wavelength. In one embodiment, the sensor is implanted and the exciting includes transcutaneously exciting fluorophores of the implanted sensor. In other embodiments, the sensor is implanted and the detecting includes transcutaneously detecting fluorophores of the implanted sensor.

In some embodiments, the method further includes transmitting signals corresponding to the detected light to a remote location. In other embodiments, the method further includes detecting light of a third wavelength. In one embodiment, the method further includes drawing fluid including the analyte from an individual, and contacting the sensor with the fluid.

In other aspects, the invention features a device that includes a detector-emitter array for detecting an analyte, the detector-emitter array including an excitation source adapted to excite a fluorophore of a sensor including fluorophores, a first detector adapted to detect fluorescence light of a first wavelength emitted by the sensor, a second detector adapted to detect fluorescence light of a second wavelength emitted by the sensor, and a third detector adapted to detect light of a third wavelength. In some embodiments, the device further includes a transmitter for transmitting a signal corresponding to light detected by the detectors to a remote location. In one embodiment, the excitation source is adapted to transcutaneously excite a fluorophore of the sensor. In other embodiments, the detector is adapted to transcutaneously detect light emitted by the sensor.

In some embodiments, the device further includes a processor for processing signals generated by the detectors.

In one embodiment, the excitation source is positioned to provide excitation radiation to a first area of the sensor and the detectors are positioned to detect light emitted from the sensor at a second area of the sensor, the first area being in a spaced apart relation to the second area.

In some embodiments, the device further includes an amplifier and an A/D converter for amplifying and digitizing the signal from the first detector. In other embodiments, the device further includes a clock for controlling a duration of a pulse emitted by the excitation source and for controlling acquisition of first data, second data, and third data from the first, second, and third detectors. In another embodiment, the device further includes a transmitter for transmitting the first data, the second data, and the third data to a remote location. In one embodiment, the device further includes an additional processor for calculating a concentration of the analyte based on the first data, the second data, and the third data.

In another embodiment, the device for detecting an analyte includes a sensor adapted to detect the analyte, the sensor including a matrix, a membrane and fluorophores, an excitation source to excite a fluorophore of the sensor, a filter device for selecting from at least one of a first filter for filtering light of a first wavelength emitted by the sensor and a second filter for filtering light of a second wavelength emitted by the sensor, a detector to detect light emitted by the sensor, and a processor for processing signals corresponding to light detected by the detector. In one embodiment, the filter device includes a liquid crystal filter tunable to the first wavelength and the second wavelength.

In another aspect, the invention features a method of detecting fluorescence emitted by a sensor using a system described herein, the method including exciting fluorophores of the sensor, detecting light of a first wavelength emitted by the sensor, and subsequently detecting light of a second wavelength emitted by the sensor. In one embodiment, the exciting includes transcutaneously exciting fluorophores of the sensor. In some embodiments, the detecting includes transcutaneously detecting light emitted by the sensor.

In other aspects, the invention features a method of correcting a measured intensity, the method including exciting a fluorophore of a sensor including fluorophores, measuring a first I(λ₁), a second I(λ₂), and a third I(λ_{B}) intensity at a first λ₁, a second λ₂, and a third λ₃ wavelength, respectively, correcting the measured intensity at the first wavelength I(λ₁) based on the intensity at the third wavelength I(λ₃) and a first set of three predetermined correction functions D(λ₁), A(λ₁), B(λ₁), and correcting the measured intensity at the second wavelength I(λ₂) based on the intensity at the third wavelength I(λ₃) and a second set of three predetermined correction functions D(λ₂), A(λ₂), B(λ₂). In one embodiment, the method further includes determining the fraction f, of the intensity due to emission by a first set of fluorescently labeled molecules D, and determining the fraction f₂ of the intensity due to emission by a second set of fluorescently labeled molecules A.

In some embodiments, the determining is based on a predetermined first I_{D}(λ₁), I_{A}(λ₁), I_{B}(λ₁), second I_{D}(λ₂), I_{A}(λ₂), I_{B}(λ₂), and third I_{D}(λ₃), I_{A}(λ₃), I_{B}(λ₃) intensity of the first set of molecules D, the second set of molecules A, and the background B at each of the first (λ₁), the second (λ₂), and the third (λ₃) wavelengths, and the first I(λ₁), second I(λ₂), and third I(λ₃) intensities normalized to the intensity at the third wavelength λ₃. In other embodiments, the determining is based on a first D(λ₁), A(λ₁), B(λ₁), second D(λ₂), A(λ₂), B(λ₂), and third D(λ₃), A(λ₃), B(λ₃) predetermined correction coefficient related to the first set of molecules D, the second set of molecules A, and the background B at each of the first (λ₁), the second (λ₂), and the third (λ₃) wavelengths. In some embodiments, the determining includes using the following equations

$${f}_{1} = - \frac{- A ( {λ}_{3} ) * B ( {λ}_{2} ) + A ( {λ}_{2} ) B ( {λ}_{3} ) + A ( {λ}_{3} ) * I ( {λ}_{2} ) - B ( {λ}_{3} ) * I ( {λ}_{2} ) - A ( {λ}_{2} ) * I ( {λ}_{3} ) + B ( {λ}_{2} ) * I ( {λ}_{3} )}{A ( {λ}_{3} ) * B ( {λ}_{2} ) - A ( {λ}_{2} ) B ( {λ}_{3} ) - A ( {λ}_{3} ) * D ( {λ}_{2} ) + B ( {λ}_{3} ) * D ( {λ}_{2} ) + A ( {λ}_{2} ) * D ( {λ}_{3} ) - B ( {λ}_{2} ) * D ( {λ}_{3} )}$$

$${f}_{2} = - \frac{B ( {λ}_{3} ) * D ( {λ}_{2} ) - B ( {λ}_{2} ) D ( {λ}_{3} ) - B ( {λ}_{3} ) * I ( {λ}_{2} ) + D ( {λ}_{3} ) * I ( {λ}_{2} ) + B ( {λ}_{2} ) * I ( {λ}_{3} ) - D ( {λ}_{2} ) * I ( {λ}_{3} )}{- A ( {λ}_{3} ) * B ( {λ}_{2} ) + A ( {λ}_{2} ) B ( {λ}_{3} ) + A ( {λ}_{3} ) * D ( {λ}_{2} ) - B ( {λ}_{3} ) * D ( {λ}_{2} ) - A ( {λ}_{2} ) * D ( {λ}_{3} ) + B ( {λ}_{2} ) * D ( {λ}_{3} )}$$

In one embodiment, the method further includes calculating the ratio of the fluorescence intensity of the first set of molecules D to the fluorescence intensity of the second set of molecules A by dividing f₁ by f₂.

In other embodiments, the method of determining the ratio of the fluorescence intensity of an energy donor D to the fluorescence intensity of an energy acceptor A of a sensor includes calculating the ratio of the donor fluorescence intensity to the acceptor fluorescence intensity is based on a first D(λ₁), A(λ₁), B(λ₁), second D(λ₂), A(λ₂), B(λ₂), and third D(λ₃), A(λ₃), B(λ₃) predetermined fluorescence coefficient of the donor D, the acceptor A, and the background B at each of first (λ₁), the second (λ₂), and the third (λ₃) wavelengths and the first I(λ₁), second I(λ₂), and third I(λ₃) intensities normalized to the third I(λ₃) intensity. In one embodiment, D is the donor fluorescence intensity due to direct excitation and A is the acceptor fluorescence intensity due to energy transfer.

In some aspects, the invention features a method of correcting for intensity associated with a background component, the method including exciting a sensor including fluorophores, measuring a first I(λ₁), a second I(λ₂), and a third intensity I(λ_{B}) corresponding to emission of the sensor at a first (λ₁), second (λ₂), and third wavelength (λ_{B}), the third wavelength (λ_{B}) being selected such that the intensity detected at the third wavelength (λ_{B}) consists of background signal, correcting the intensity at the first wavelength I(λ₁) based on the third intensity I(λ_{B}) and a first predetermined correction function B(λ₁), and correcting the intensity at the second wavelength I(λ₂) based on the third intensity I(λ_{B}) and a second predetermined correction function B(λ₂). In some embodiments, the method further includes calculating the ratio of the corrected intensity at the first wavelength (λ₁) to the corrected intensity at the second wavelength (λ₂). In other embodiments, the property is the concentration of the analyte. In one embodiment, the background signal is due to skin. In another embodiment, the first wavelength is 600 nm, the second wavelength is 700 nm, and the third wavelength is 565 nm.

The invention features a system and device for noninvasively detecting an analyte in an individual, e.g., a human or other mammal.

The system and device can be configured to transcutaneously excite an implanted sensor and detect analyte detected by the sensor. Alternatively, or in addition, the system and device can be configured to directly excite a sensor and detect analytes in in vitro samples where the sensor is disposed in the in vitro sample. The system or a component of the system can be placed in close proximity to (e.g., in contact with, or above) the skin of the individual in the area of the implanted sensor.

Other features and advantages will be apparent from the following description of the preferred embodiments and from the claims.

### Glossary

In reference to the invention, these terms have the meanings set forth below as used herein:

The term “transcutaneous” refers to transmission through any layer of the skin including the dermis, epidermis, and combinations thereof.

The term “fluorescence” refers to radiation emitted in the ultraviolet, visible and infrared regions of the electromagnetic spectrum in response to excitation by radiation of a particular wavelength. It includes both short-lived (nanosecond) and long-lived excited state lifetimes; the latter is sometimes referred to as phosphorescence.

The term “dichromatic mirror” refers to a mirror (e.g., a dichroic mirror) having the ability to transmit a first set of wavelengths and to reflect a second set of wavelengths.

### DRAWINGS

FIG. 1 illustrates a schematic representation of one embodiment of a system for detecting analyte.

FIG. 2 illustrates a schematic representation of a system for detecting analyte according to a second embodiment.

FIG. 3 illustrates a schematic representation of a system for detecting analyte according to a third embodiment.

FIG. 4 illustrates an embodiment of a system for detecting analyte that includes a semiconductor chip that includes an excitation source and detectors.

FIGS. 5A and 5B illustrate two embodiments of a system for detecting analyte that includes optical fibers.

FIG. 6 illustrates an embodiment of a system for detecting analyte that includes a single mode optical fiber.

FIG. 7 illustrates an embodiment of a system for detecting analyte that includes a single mode optical fiber and a fiber optic bundle.

FIG. 8 illustrates another embodiment of a system for detecting analyte that includes a single mode optical fiber and a fiber optic bundle.

FIG. 9A is a block diagram showing a single photon counting system for detecting analyte concentration, according to one embodiment.

FIG. 9B is a block diagram showing an analog signal detection system for detecting analyte concentration, according to another embodiment.

### DETAILED DESCRIPTION

The system for detecting and analyzing (e.g., quantifying) analyte in an individual includes a sensor for detecting the presence of the analyte, an excitation source for exciting fluorophores located in the sensor, at least one detector for detecting light emitted by the sensor, a means for transferring the signals associated with the detected light to a device capable of processing the signals, a processor for processing the signals received from the detector(s) and, optionally, correlating the signals to a property (e.g., concentration) of the detected analyte (e.g., glucose) and a display for displaying information related to the signals, e.g., a property of the analyte. The system can be configured to transcutaneously excite the fluorophores in an implanted sensor, transcutaneously detect light emitted by an implanted sensor (e.g., the fluorescence emitted by the fluorophores of the sensor), or a combination thereof. Alternatively, the system can be configured to be capable of directly exciting the fluorophores of a sensor, e.g., the sensor is not implanted, and directly detecting the light emitted by the sensor.

The system can be used to detect a variety of analytes including, e.g., carbohydrates (e.g., glucose), glycoproteins, glycopeptides, enzymes, glycolipids, hormones, lipoproteins, antibodies, antigens, haptens, steroids, theophylline, creatinine, drugs, nucleotides, polynucleotides, pesticides, and combinations thereof.

The system can include any sensor suitable for detecting analytes. The sensor includes a fluorescence reagent that is capable of detecting the presence of an analyte. The fluorescence reagent includes at least one fluorophore and optionally a component labeled with a fluorophore. One example of a useful sensor construction includes a core that includes a polymer matrix, e.g., a hydrogel, and a fluorescence reagent disposed in the polymer matrix, and a semipermeable coating (i.e., membrane) surrounding the core. The fluorescence reagent is preferably mobile within the polymer matrix. The sensor can be constructed to be suitable for implantation, for ex vivo use, or for both implantation and ex vivo use. Sensors that are to be implanted in a host preferably include an exterior coating that is biocompatible.

Alginate is one example of a useful hydrogel polymer matrix. Preferred alginate gels are derived from alginate that includes blocks of 1,4-linked (D-mannuronic acid) (M) and (-1-glucoronic acid) (G) linked together, e.g., in alternating MG blocks. Preferred alginate includes a high G block content, e.g., at least about 60% G block. As the percentage of G blocks in the alginate composition increases, the pore size and the strength of the resulting gel matrix increases. Alginate gels having a high M block content appear to be more immunogenic relative to gels having a high G block content.

Other suitable hydrogels include, e.g., carrageenan, gum, e.g., xanthan gum, agarose, agar, collagen, gelatin, chitosan, polyethylene glycol, and polyethylene oxide gels, and combinations thereof. Useful polymer matrices include, e.g., polyacrylamide, polyacrylate, and polymethacrylate gels, and combinations thereof.

The semipermeable coating is a porous polymer coating that can be prepared from a variety of polymers including, e.g., heteroploymers, homopolymers and mixtures thereof. The permeability of the coating is such that the analyte of interest flows in and out of the sensor at a physiologically relevant rate, the reagents within the sensor remain within the sensor (i.e., the host is not exposed to the reagents), the analyte of interest comes into contact with the reagent, and components of a predetermined molecular weight are inhibited, and preferably prevented, from entering the sensor. The type and molecular weight of the polymer from which the semipermeable coating is prepared and the thickness of the coating are selected to provide the desired permeability. Suitable semipermeable coatings include polymers such as polylysine and polyornithine, as well as crosslinked gels including, e.g., crosslinked hydrogels (e.g., alginate gel and agarose gel), polyethylene oxide, polystyrene sulfonic acid.

Various semipermeable coatings are useful including semipermeable coatings prepared from polydisperse polymers, monodisperse polymers and combinations thereof. One useful class of polydisperse polymers has an average molecular weight of from about 4 kiloDaltons (kDa) to about 18 kDa, from about 8 kDa to about 12 kDa, from about 9 kDa to about 10 kDa, or even about 9.4 kDa and a polydispersity index Mn/Mw (dI) greater than 1, from greater than 1.0 to about 1.5, or even from about 1.1 to 1.4.

Examples of useful polymers for forming the semipermeable coating include polyamino acids (e.g., polylysine and polyornithine), polynucleotides, and combinations thereof. Suitable polymers include, e.g., polyamino acids having a length of from 19 to 60 amino acids, from 38 to about 60 amino acids, or even from about 43 to about 48 amino acids. Suitable polydisperse polyamino acids are available from Sigma Chemical Company (St. Louis, Mo.).

The semipermeable coating can include a mixture of monodisperse polymers of different molecular weights.

The semipermeable coating can include multiple layers in which each layer is prepared from the same polymer composition or a different polymer composition. For example, the semipermeable coating can include one or more layers of polydisperse polymers, monodisperse polymers, and combinations thereof. Useful monodisperse polymers include monodisperse polyamino acids including, e.g., poly-L-lysine monodisperese homopolymers having 33, 47 and 60 residues. In some cases, although multiple layers have been applied to the sensor, the individual layers may not be individually discernable.

Preferably the semipermeable coating excludes IgG and complement (e.g., complement C1q). Preferably the semipermeable coating excludes molecules having a molecular weight greater than 100 kDa, greater than 60 kDa, greater than 30 kDa, greater than 10 kDa, or even greater than 3 kDa from entering the sensor.

The composition of the semipermeable coating can be selected to reduce the volume of the core. Coating compositions that include relatively low molecular weight polydisperse polyamino acid (e.g., a polylysine or polyornithine) can significantly reduce the volume of the gel core to which it is applied. In many cases the reduction in volume is at least about 50%, at least 60%, or even at least 70%. Useful polyamino acids have a molecular weight no greater than about 30 kDa, no greater than about 15 kDa, no greater than about 10 kDa, no greater than about 8 kDa, no greater than about 7 kDa, no greater than about 5 kDa, no greater than about 4 kDa, no greater than about 3 kDa, or even no greater than about 1.5 kDa.

Polydisperse polylysine having a molecular weight of 3 kDa, 7 kDa, 9.6 kDa, or even 12 kDa, can result in a significant reduction (approximately 30% in some cases) in the diameter of the core to which the coating it is applied.

The low molecular weight polyamino acid also forms a coating having good permselective properties and can produce a surface that is “pruned” or crenellated, i.e., relatively convoluted or rough. Such pruned surfaces may elicit a fibrotic response. The application of alginate to the pruned surface can provide a relatively smooth surface on the exterior of the sensor, which inhibits fibrosis and reduces light scattering effects.

Implantable sensors preferably include an exterior surface that is sufficiently biocompatible so as not to induce a fibrotic response from the host's immune system that will impair or prevent the diffusion of the analyte of interest into and out of the sensor at a physiologically relevant rate, while being sufficiently nonbiocompatible so as allow the host to form a sheath around the sensor to maintain the sensor in position in the host. Suitable biocompatible coating compositions include the compositions suitable for the polymer matrix of the core including, e.g., hydrogels (examples of which include alginate and agarose).

Useful methods of providing immunoisolating coatings are described, e.g., in U.S. Pat. No. 6,126,936.

One example of a useful class of fluorescence reagents includes those reagents that are based on non-radiative fluorescence resonance energy transfer (“FRET”). FRET generally involves the non-radiative transfer of energy between two fluorophores, one an energy donor (“D”) and the other an energy acceptor (“A”). Any appropriately-selected donor-acceptor pair can be used for a FRET-based sensor, provided that the emission of the donor overlaps with the excitation spectra of the acceptor and both members can absorb light energy at one wavelength and emit light energy of a different wavelength. FRET is further described in U.S. Pat. No. 5,342,789 and incorporated herein.

Useful sensors are described, e.g., in U.S. patent application Ser. Nos. ______ and ______ entitled, “SEMIPERMEABLE SENSORS FOR DETECTING ANALYTE,” filed on Oct. 31, 2003 (Attorney Docket Number 205-009US1), and incorporated herein. Other suitable sensors are described, e.g., in U.S. Pat. Nos. 6,040,194 and 6,625,479, and U.S. Patent Application Serial Nos. 2002010279 and 2002043651.

The wavelengths at which emission radiation is detected are optimized for the reagent of the sensor. For sensors based on FRET, for example, the wavelengths are typically selected to include the emission maxima of the donor and acceptor spectrum. In one embodiment, the background wavelength (λ_{B}) is 565 nm, the donor wavelength (λ₁) is 600 nm and the acceptor wavelength (λ₂) is 700 nm. In other embodiments, other wavelengths are used.

The following table lists examples of suitable dyes (by trade designation and vendor) and the approximate emission maximum (i.e., useful region of measurement) in nanometers (nm).

TABLE 1

| | | Approximate Emission |
|---|---|---|
| | | Maximum or region of |
| Dye | Vendor | measurement in nm |
| Alexa 546 | Molecular Probes¹ | 573 |
| Alexa 555 | Molecular Probes | 565 |
| Alexa 568 | Molecular Probes | 603 |
| Alexa 594 | Molecular Probes | 617 |
| Alexa 610 | Molecular Probes | 628 |
| Alexa 633 | Molecular Probes | 647 |
| Alexa 647 | Molecular Probes | 665 |
| Alexa 660 | Molecular Probes | 690 |
| Alexa 680 | Molecular Probes | 702 |
| Alexa 700 | Molecular Probes | 723 |
| Alexa 750 | Molecular Probes | 775 |
| Bodipy630/650 | Molecular Probes | 640 |
| Bodipy 650/665 | Molecular Probes | 660 |
| Cy 3 | Amersham BioSciences² | 570 |
| Cy 3B | Amersham BioSciences | 572 |
| Cy 3.5 | Amersham BioSciences | 596 |
| Cy 5 | Amersham BioSciences | 670 |
| Cy 5.5 | Amersham BioSciences | 694 |
| Cy 7 | Amersham BioSciences | 767 |
| Oyster 556 | DeNovo³ | 570 |
| Oyster 645 | DeNovo | 666 |
| Oyster 656 | DeNovo | 674 |

¹Molecular Probes, Eugene, Oregon.
²Amersham BioSciences, Cardiff Wales.
³DeNovo Biolabels GmbH, Munster, Germany.

For sensors based on a true chromatic shift, i.e., binding of the analyte causes a true chromatic shift, then the wavelengths are preferably selected to be the initial wavelength (i.e., the wavelength at which the sensor chemistry (i.e., the reagents of the sensor) emits in the absence of analyte binding) and the shifted wavelength (i.e., the wavelength at which the sensor chemistry emits in the presence of analyte binding). Sensor chemistries can also exhibit an intensity shift in the presence of analyte binding. For such sensor chemistries, the wavelengths can be selected based on a first wavelength at which an intensity increase occurs when analyte binding occurs and a second wavelength at which the intensity is independent of analyte binding.

The excitation source can be any source capable of exciting fluorophores of the sensor. The excitation source is optionally configured to transcutaneously excite fluorophores in implanted sensors. Suitable excitation sources include lasers, light emitting diodes (LED), gas discharge lamps and incandescent lamps. The excitation source can be located on a semiconductor chip or wafer, i.e., a semiconductor light-emitting device. The light emission element can be selected to emit light of wavelengths suitable for exciting the fluorophore of a predetermined sensor. In one embodiment, the excitation source is a light emitting diode that emits light having wavelengths from about 460 nanometers (nm) to about 590 nm, or even 530 nm to about 560 nm including, e.g., blue, green, yellow and red LEDs. Suitable semiconductor light emission element are available at www.nichia.com.

There are multiple Sources of suitable diode lasers. Including, e.g.,

| Wavelength nm | Manufacturer |
|---|---|
| 370-380 | Nichia Corp. Tokushima, Japan |
| 440-415 | Nichia Corp. Tokushima, Japan |
| 435-445 | Nichia Corp. Tokushima, Japan |
| 532 | BWTech, Newark, Delaware |
| 635 | US Lasers, Hazelehurst, GA |
| 645 | US Lasers, Hazelehurst, GA |
| 650 | US Lasers, Hazelehurst, GA |
| 655 | US Lasers, Hazelehurst, GA |
| 660 | US Lasers, Hazelehurst, GA |
| 670 | US Lasers, Hazelehurst, GA |

Suitable light emitting diodes commercially available from Nichia Corp. include, e.g.,

| Type | Wavelength Range nm |
|---|---|
| W | 565-475 |
| C | 495-500 |
| D | 500-505 |
| E | 505-510 |
| F | 510-520 |
| G | 520-535 |
| H | 535-545 |
| K | 573-577 |
| R | 615-635 |

The excitation source can be pulsed at a predetermined repetition rate. The rate at which data is collected is determined by parameters including the rate of pulsing, the duration of the pulses and the intensity of the pulses. The excitation source is preferably pulsed and the detector(s) is preferably phase-locked with the pulse of the excitation source to reduce and preferably eliminate interference that may arise from ambient light.

The detector is capable of detecting fluorescence. The detector is optionally capable of transcutaneously detecting fluorescence emitted by fluorophores in an implanted sensor. The detectors of the system preferably are configured to measure light intensity at predetermined wavelengths corresponding to the reagent chemistry of the sensor. Where the system is to detect fluorescence emitted by an implanted sensor, the system can include two detectors capable of measuring at two different wavelengths associated with the sensor, and a third detector capable of measuring at a wavelength associated with the fluorescence emitted by the skin of the individual in which the sensor is implanted, as well as the light scattering caused by the skin. The third detector can provide signals that enable correction of the background signal and/or noise associated with the system. The detectors can be configured to collect emitted light simultaneously.

The mechanism that collects the light emitted by the sensor for transmission to the detector preferably is spaced (e.g., translationally or rotationally) from the excitation radiation received by the sensor such that the amount of excitation radiation collected by the detector due to light scattering is minimized, as is the amount of autofluorescence of the skin in the region of excitation (i.e., the area of the skin that is exposed to the excitation radiation).

Useful detectors include, e.g., photodiodes (e.g., silicon photodiodes and avalanche photodiodes), photoresistors, photomultiplier tubes, and charge coupled devices. The detectors can also be an array of detectors including, e.g., photodiode array detectors and charge coupled array detectors. Various detectors suitable for use with the present invention, including photomultipliers, photodiode arrays, avalanche photodiodes, and charge-coupled device arrays, are available from Hamamatsu Corporation USA located in Bridgewater, N.J.

The detectors can be configured to detect single photons and to operate in a single photon counting mode. Useful detectors for single photon counting include photomultiplier tubes, photodiodes, and avalanche photodiodes.

The detector can be configured to produce an analog signal or a digital signal in response to light detected by the detector and can include an analog to digital converter.

The detectors can include circuits for amplifying signals (e.g., operational amplifiers), discriminating between signals and background noise, and converting signals generated by the detector to TTL pulses. The signal generated by the detector is preferably amplified, discriminated and/or digitized.

The intensity of the fluorescence (i.e., signal) detected by the detector can be measured directly or accumulated in data buffers. The detectors and/or the circuitry associated with the detectors can be configured to function in a variety of ways including, e.g., to count the number of photon pulses detected over a fixed time interval, to calculate the period of time required to obtain a predetermined number of photon pulses (e.g., counting the number of clock pulses that occur before the number of photon pulses reaches a predetermined value), to determine the average number of clock pulses that occur between photon events, and combinations thereof.

The spectral selectivity of a detector can be achieved by providing a filter in the path of light received by the detector such that light passes through the filter prior to reaching the detector. The detector can be made to be specific for a particular wavelength of light or band of wavelengths through the selection of one or more filters. A silicon photodiode detector, for example, can be constructed to include deposited thin-film bandpass filters, long pass filters, and combinations thereof. Suitable filters include interference filters, band pass filters, light absorbing film, diffraction gratings, and prisms. Preferred band pass filters are capable of separating the excitation radiation emitted by the excitation source from the fluorescence emission radiation emitted by the fluorophores of the sensor. Other suitable filters include liquid crystal based filter arrays, which are tunable to a desired wavelength range, as well as arrays of tunable filters. Suitable liquid crystal-based filter arrays are commercially available from Cambridge Research and Instruments, Inc. (Cambridge, Mass.). The filter is selected according to the desired wavelength selectivity. Suitable filters include filters suitable for use with the dyes set forth above in Table 1. Useful filters are available from Omega Optical (Brattleboro, Vt.).

Alternatively or in addition to filters, the detectors can be made from materials that can be adapted, e.g., tuned, to be sensitive to a particular wavelength of light. For example, the photosensitive elements could be tuned to sense, for example, fluorescence emission radiation and to substantially exclude excitation radiation from the excitation source. In this regard, photoresistive detectors can be chemically tuned to be sensitive substantially at a specific wavelength, thereby reducing or eliminating the need for a separate filter element. Suitable photosensitive elements are commercially available including, e.g., devices available from Silonex Inc. (Montreal, Quebec, Canada) where peak wavelength sensitivity is adjusted and optimized based on varying ratios of dopants and mix ratios within a cadmium sulfide base.

The process for storing and analyzing the signals generated by the detectors can be implemented using one or more computers, controllers, or processors. The signals received by the data processor are processed to provide information related to a property of the detected analyte including, e.g., the presence or absence of the analyte, concentration of the analyte in the fluid of interest, concentration of analyte in the mammal, and the rate of change of analyte concentration. The processor can also provide information to an individual or a device regarding an activity related to the property of the analyte including, e.g., instructions as to the amount of medicament that should be administered, providing a notice such as an alarm if a predetermined condition is met (e.g., the amount of analyte is at a level that is critical to the individual) such as hypoglycemia in a diabetic mammal, and graphic and tabular information related to time and concentration of the analyte detected. The instructions to the device can include, e.g., instructions to provide a predetermined amount of medicament (e.g., the appropriate amount of insulin) via a medication dispenser, e.g., an external or internal pump (e.g., an insulin infusion pump).

In one embodiment, the data processor receives data from two detectors that are detecting light intensity at two different wavelengths and calculates the ratio of the intensity of the signals received at each detector. From the ratio, the processor can calculate various properties of the system including the concentration of the analyte of interest. The concentration may be determined using an experimentally-derived correlation equation or look-up table relating this ratio to a given analyte. In various other embodiments, the concentration of a given analyte may be determined by any measure of fluorescence. In other embodiments, the concentration of a given analyte can be determined by any measure of energy transfer, as indicated by the fluorescence detected by the detector. For example, in one embodiment, the concentration is determined based on the energy transfer efficiency. In another embodiment, the data processor receives data from at least one detector and determines the rate of decay of the fluorescence, which is also referred to as the fluorescence excited state lifetime.

In one embodiment, the system includes the ability to correct for background signal present in the signals received at the detectors. In this embodiment, the system includes the ability to detect the background signal, i.e., the portion of the signal not attributable to the sensor chemistry. For an implanted sensor, the background signal may include the autofluorescence and the light scattering generated by background components including, e.g., skin, components of the sensor other that the sensor chemistry, and components of the measurement system. For an ex vivo sensor, the background signal may include the autofluorescence and light scattering generated by components of the sensor other that the sensor chemistry, and components of the measurement system.

In one embodiment, for example, the system may be adapted to detect fluorescence associated with human skin. In this embodiment, the system may be adapted to detect any wavelength, λ_{B}, where skin (or other source of a background signal) emits, but the sensor chemistry, e.g., the dyes present in the sensor, does not, such that the intensity of the signal at this wavelength, λ_{B}, is only associated with the skin.

In one embodiment, the intensity of the background signal is used to obtain a background corrected fluorescence emission spectrum, BKCorreI(λ), of the sensor chemistry by measuring the intensity of the sensor chemistry as a function of the wavelength of the signal, I(λ), and measuring the intensity, I(λ_{B}), of the background signal. The correction function, B(λ), is the spectrum of the background normalized to λ_{B} such that B(λ_{B})=1. B(λ) is obtained in a calibration step by taking a spectrum of the background, which spectrum is then stored in the system. The term spectrum as used herein refers to any number of wavelengths. As indicated above, the background spectrum is preferably obtained on all of the background components of the system except the sensor chemistry, but it can be limited to one or more of the background components. Accordingly, BKCorreI(λ)=I(λ)−I(λ_{B})B(λ). When measuring the spectrum at three wavelengths λ₁ λ₂ λ_{B}, the three simultaneous equations are as follows:

BKCorreI(λ_{B})=I(λ_{B})−I(λ_{B})1;

BKCorreI(λ₁)=I(λ₁)−I(λ_{B})B(λ₁); and

BKCorreI(λ₂)=I(λ₂)−I(λ_{B})B(λ₂).

These three equations may be used to derive an intrinsic parameter of the sensor. An intrinsic parameter of the sensor is a parameter that is dependent upon a property of the analyte, e.g., the concentration of the analyte, but which is not dependent upon extrinsic factors such as illumination intensity, sensor volume, and sensor geometry. Examples of such intrinsic properties include the ratio of the corrected intensity at λ₁ to the corrected intensity at λ₂

$$\frac{BKCorrel ( {λ}_{1} )}{BKCorrel ( {λ}_{2} )}$$

and the ratio

$$\frac{BKCorrel ( {λ}_{1} ) - BKCorrel ( {λ}_{2} )}{BKCorrel ( {λ}_{1} ) + BKCorrel ( {λ}_{2} )} .$$

For sensors based on fluorescence resonance energy transfer, further examples of such intrinsic properties include the transfer efficiency, the fraction of donor quenching, and the fraction of acceptor emission.

According to another embodiment, a parameter of the system is calculated after separating out the background component from the overall raw signal. The raw signal is a composite of three signals, namely the signals from molecules (D) of the sensor chemistry that are bound to the analyte of interest, molecules (A) of the sensor chemistry that are not bound to the analyte of interest, and the background (B). The emission spectrum of each of the D, A, and background (B) are obtained. Each of these spectra is then normalized to a wavelength λ₁ by dividing each spectrum by its value at λ₁ to generate normalized spectra, which are identified as D(λ) for the D molecules, A(λ) for the A molecules, and B(λ), for the background, and then stored. The normalized fluorescence emission spectrum, I(λ), of a sensor is calculated by obtaining the spectrum of the sensor F(λ) and then dividing the spectrum by the value at F(λ₁). If f₁, f₂, and f₃ are the fractions of the three normalized spectra in the normalized measured signal from the sensor, then there are three equations that must be solved simultaneously,

I(λ₁)=f₁D(λ₁)+f₂A(λ₁)+f₃B(λ₁),

I(λ₂)=f₁D(λ₂)+f₂A(λ₂)+f₃B(λ₂),

I(λ₃)=f₁D(λ₃)+f₂A(λ₃)+f₃B(λ₃).

Because of the normalization condition, the first of these equations is equivalent to:

1=f₁+f₂+f₃

These three equations may be solved for f₁, f₂, and f₃. The solution provides the number of molecules D of the sensor chemistry bound to the analyte of interest and the number of molecules A not bound to the analyte and can be determined as follows

$${f}_{1} = - \frac{- A ( {λ}_{3} ) * B ( {λ}_{2} ) + A ( {λ}_{2} ) B ( {λ}_{3} ) + A ( {λ}_{3} ) * I ( {λ}_{2} ) - B ( {λ}_{3} ) * I ( {λ}_{2} ) - A ( {λ}_{2} ) * I ( {λ}_{3} ) + B ( {λ}_{2} ) * I ( {λ}_{3} )}{A ( {λ}_{3} ) * B ( {λ}_{2} ) - A ( {λ}_{2} ) B ( {λ}_{3} ) - A ( {λ}_{3} ) * D ( {λ}_{2} ) + B ( {λ}_{3} ) * D ( {λ}_{2} ) + A ( {λ}_{2} ) * D ( {λ}_{3} ) - B ( {λ}_{2} ) * D ( {λ}_{3} )}$$

$${f}_{2} = - \frac{B ( {λ}_{3} ) * D ( {λ}_{2} ) - B ( {λ}_{2} ) D ( {λ}_{3} ) - B ( {λ}_{3} ) * I ( {λ}_{2} ) + D ( {λ}_{3} ) * I ( {λ}_{2} ) + B ( {λ}_{2} ) * I ( {λ}_{3} ) - D ( {λ}_{2} ) * I ( {λ}_{3} )}{- A ( {λ}_{3} ) * B ( {λ}_{2} ) + A ( {λ}_{2} ) B ( {λ}_{3} ) + A ( {λ}_{3} ) * D ( {λ}_{2} ) - B ( {λ}_{3} ) * D ( {λ}_{2} ) - A ( {λ}_{2} ) * D ( {λ}_{3} ) + B ( {λ}_{2} ) * D ( {λ}_{3} )}$$

$${f}_{3} = - \frac{A ( {λ}_{3} ) * D ( {λ}_{2} ) - A ( {λ}_{2} ) D ( {λ}_{3} ) - A ( {λ}_{3} ) * I ( {λ}_{2} ) + D ( {λ}_{3} ) * I ( {λ}_{2} ) + A ( {λ}_{2} ) * I ( {λ}_{3} ) - D ( {λ}_{2} ) * I ( {λ}_{3} )}{A ( {λ}_{3} ) * B ( {λ}_{2} ) - A ( {λ}_{2} ) B ( {λ}_{3} ) - A ( {λ}_{3} ) * D ( {λ}_{2} ) + B ( {λ}_{3} ) * D ( {λ}_{2} ) + A ( {λ}_{2} ) * D ( {λ}_{3} ) - B ( {λ}_{2} ) * D ( {λ}_{3} )}$$

The ratio of the signal due to D to the signal due to A can then be calculated as f₁/f₂.

The predetermined values for D(λ₁), D(λ₂), D(λ₃), A(λ₁), A(λ₂), A(λ₃), B(λ₁), B(λ₂), and B(λ₃) are obtained from an available look-up table. The values for f₁, f₂, and f₃, and the ratio, R, or other intrinsic parameters, may then be calculated using the above formulas. A look-up table showing analyte concentration as a function of R may then be used to determine the analyte concentration. Where the sensor chemistry is based on FRET and D is the donor and A is the acceptor, the values for the acceptor fluorescence are those for energy-transfer based excitation, not direct excitation.

The above-discovered relationship can also be used to calculate other parameters of the system including, for energy transfer-based sensors, e.g., transfer efficiency, fraction donor quenching, and fraction sensitized acceptor emission.

The system optionally includes a telemetry system for transmitting signals from one or more of the detectors to a remote location. By “remote” it is meant not physically connected so as to receive a signal through a wire or other mechanical means. Such remote locations include, e.g., a station remote from the user, a receiver in a readout device constructed to be worn by a user, and a read out device to be held by the user. The telemetry system includes a transmitter and a receiver and can employ any suitable telemetry means for transmitting data on a real time basis to a remote location including, e.g., radio frequency and infrared telemetry means. Data can be transmitted and received either in an analog mode (e.g., via frequency or amplitude modulation of a carrier radio frequency signal) or as a digitally encoded signal.

Several variables can be simultaneously transmitted to the processor using different frequencies using several transmitters. Alternatively, a single transmitter having multiple channels can transmit a combined signal to a receiver, with the signal being subsequently decoded, separated into its multiple individual parts, filtered and regenerated as the individual original signals corresponding to the individual signals generated at each detector.

The telemetry system can be activated according to a variety of mechanisms and under a variety of conditions. For example, the telemetry system can be continuously on and can perform a function driven by a clock, or the telemetry system can be off and then activated by a command from a component of the system when a reading is to be taken.

According to various embodiments of the present invention, some or all processing of data from the sensors is performed at the detection site. In one embodiment, for example, each pulse detected by each of the detectors is transmitted in real time for remote processing. In another embodiment, a total count for each detector, or a sum of the intensity of the pulses, is calculated locally and this total or sum is then transmitted for further remote processing. In yet another embodiment, all processing including calculation of analyte concentration is performed locally (i.e., not at a remote location). This concentration is then transmitted for further remote processing or response.

Information related to the detected signals can be displayed on any suitable display including, e.g., computer screen, video screen, hand-held devices (e.g., palm display devices (e.g., personal digital assistant), telephones and pagers), and chart recorders. The information provided by the display can be provided in the form of a digital display on the device, itself, a remote display, a printout at a remote or attached device, and combinations thereof. The information displayed can include, e.g., the information and instructions provided by the processor as described herein.

In other configurations, the processor stores the data on a removable medium that can be removed from the system or a component of the system. The removable medium can then be provided to another entity including, e.g., a physician or a device for reading information on the removable medium. The removable medium can be capable of insertion into another device, which can read the medium and optionally analyze the information, modify the information, provide the information to another device, and combinations thereof. Examples of such removable media include, e.g., removable memory components (e.g., data cartridges, magnetic or optical recording discs, magnetic tape, flash memory, or any other type or removable storage device known in the art).

A variety of passive, active, and inductive power sources can be used to power the system including the various components of the system. The power supply may consist of battery (e.g., micro batteries), solar powered cells, inductive power link, energy from biological sources, micro power units, hydrogen fuel cells, and fuel cells that use glucose and oxygen as energy sources. Any other power sources known in the art may also be used to power the system. One suitable power source operates by induction.

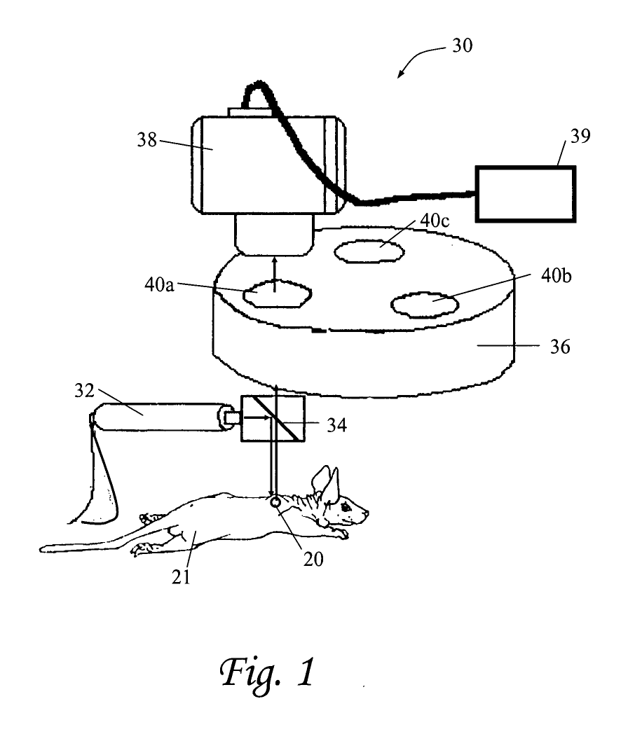

A variety of configurations of the system for detecting analyte are contemplated. FIG. 1 illustrates an embodiment of a device 30 for detecting analyte that includes an excitation source 32 (e.g., a laser), a dichromatic mirror 34, a filter device 36 capable of sequentially providing filters of at least two different wavelengths and a detector 38. In operation, light emitted by the excitation source 32 travels to the dichromatic mirror 34 where it is reflected toward a sensor 20 implanted in a mouse 21. The sensor 20 includes fluorophores. The dichromatic mirror 34 is selected such that the wavelength of light emitted by excited fluorophores in the sensor 20 passes through the dichromatic mirror 34, through a filter 40 on the filter wheel 36 to the detector 38. The filter device 36 includes a number of filters 40a, 40b, 40c capable of filtering different wavelengths of light. After fluorescence data is obtained using the first filter 40a positioned in the path of the emitted fluorescence light, the filter wheel 36 is rotated such that light emitted by the excited sensor 20 passes through the second filter 40b to the detector 38. After fluorescence data is obtained the filter wheel 36 is rotated again such that light passes through the third filter 40c to the detector 38. The signals generated by the detector in response to the fluorescence emitted by the excited sensor, and optionally background components, are transmitted to the data processor 39 where one or more properties of the detected analyte are determined.

In the various configurations of the system, the dichromatic mirror(s) can be replaced with a partially reflecting mirror(s) capable of reflecting a first portion of the light and transmitting a second portion of the light.

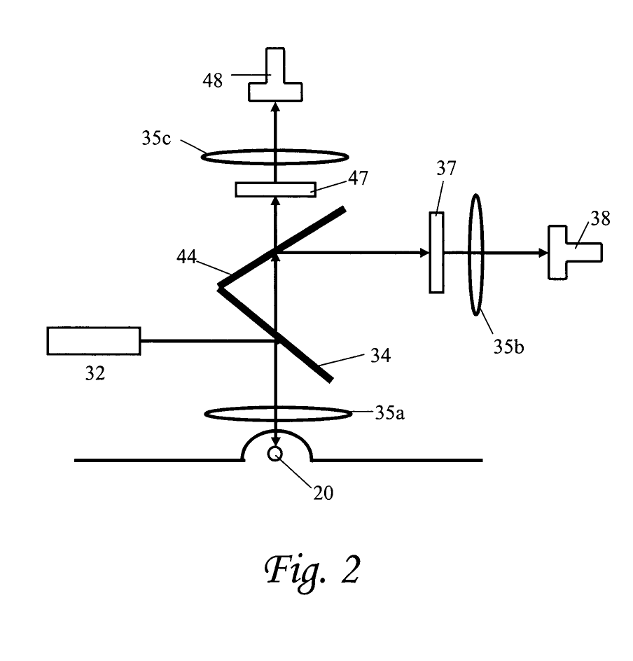

FIG. 2 illustrates an embodiment of the device that includes two detectors 38, 48, two dichromatic mirrors 34, 44, and two filters 37, 47. In operation, light emitted by the excitation source 32 travels to the first dichromatic mirror 34, which is positioned at a suitable angle (e.g., 45 degree angle) to the light emitted by the excitation source, and is reflected toward a sensor 20. The light travels through a focusing lens 35a focused at the sensor 20 prior to reaching the sensor 20. The dichromatic mirror 34 is selected such that the wavelength of light emitted by excited fluorophores in the sensor passes through the dichromatic mirror 34, to the second dichromatic mirror 44. The second dichromatic mirror 44 is configured to reflect light having a first wavelength λ₁ shorter than a second wavelength λ₂ and to transmit light having a wavelength at least as long as the second wavelength λ₂. The second dichromatic mirror 44 is positioned at a 45 degree angle to the path of incident light transmitted from the first mirror 34. The light reflected from the second dichromatic mirror 44 passes through the first filter 37, through a focusing lens 35b to the first detector 38. The light transmitted through the second dichromatic mirror 44 passes through the second filter 47, through a focusing lens 35c, to the second detector 48.

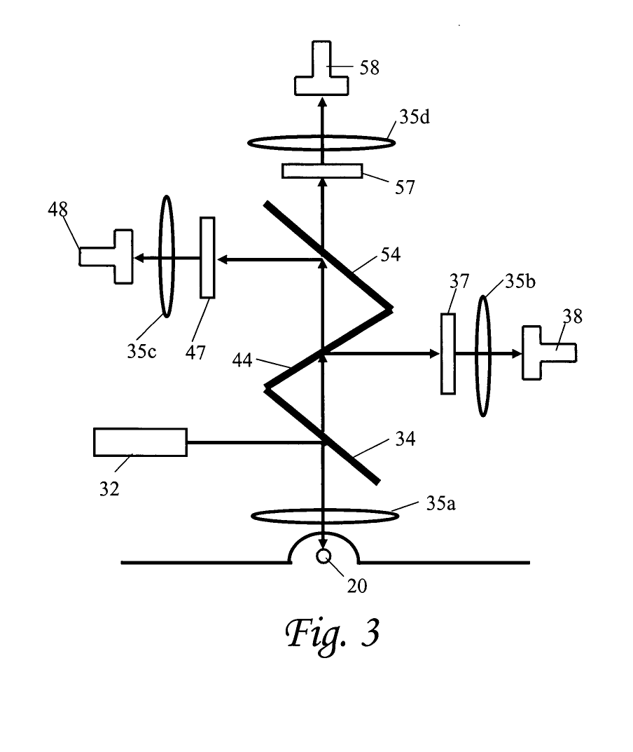

FIG. 3 illustrates an embodiment of the device that includes three detectors 38, 48, 58, three dichromatic mirrors 34, 44, 54 and three filters 37, 47, 57. In operation, light emitted by the excitation source 32 travels to the first dichromatic mirror 34, which is positioned at a 45 degree angle to the light emitted by the excitation source 32 where it is reflected toward a sensor 20 and passes through a focusing lens 35a to the sensor 20. The first dichromatic mirror 34 is selected such that the wavelength of light emitted by excited fluorophores in the sensor passes through the dichromatic mirror 34, to the second dichromatic mirror 44. The second dichromatic mirror 44 is configured to reflect light having a wavelength λ₁ shorter than a second wavelength λ₂ and to transmit light having a wavelength at least as long as the second wavelength λ₂. The second dichromatic mirror 44 is positioned at a 45 degree angle to the path of light passing from the first mirror 34. The light reflected from the second dichromatic mirror 44 passes through the first filter 37, through focusing lens 35b, to the first detector 38. The light transmitted through the second dichromatic mirror 44 passes to the third dichromatic mirror 54 where light having a wavelength shorter than λ₃ is reflected and passes through filter 47, through a focusing lens 35c, to a second detector 48, and light having a wavelength at least as long as wavelength λ₃ passes through the third dichromatic mirror 54, through the third filter 57, through a focusing lens 35d, and to the third detector 58.

Alternatively, the system can be configured such that a portion of the light passes through the first dichromatic mirror to a first detector, a second portion of the light is reflected by the first dichromatic mirror and passed to a second dichromatic mirror where a third portion of the light is reflected to a second detector and a fourth portion of the light is passed through the dichromatic mirror to a third detector.

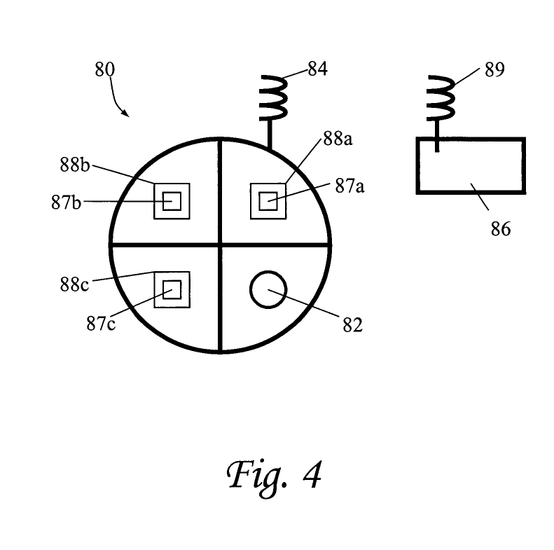

FIG. 4 illustrates an embodiment of the device in which an optoelectronic chip 80 includes an excitation source 82 and three detectors 88a, 88b, 88c. In one embodiment, the source 82 and the detectors 88 are formed on separate wafers and physically coupled to form an integral chip 80. The chip 80 may further include additional components for processing and transmitting information received from the detectors 88. Thin film filters 87a, 87b, 87c specific for different wavelengths λ₁, λ₂, λ₃ are positioned on the detectors 88a, 88b, 88c, respectively, such that light passes through filters 87a, 87b, 87c prior to reaching the detectors 88a, 88b, 88c, respectively. A filter can optionally be positioned over the excitation source such that light emitted by the excitation source passes through the filter. In operation, the chip 80 is placed over a sensor such that the excitation source 82 is capable of exciting (e.g., transdermally exciting) a fluorophore in the sensor and the detectors 88a, 88b, 88c are positioned to detect light emitted by the excited fluorophores and, where applicable, the wearer's skin. The optoelectronic chip 80 can include an antenna 84 for transmitting the signals generated by the detectors 88a, 88b, 88c to a remote processor 86 that includes a receiver 89. One or more of the detectors, the excitation source, and the transmitting antenna can be located on one or more separate chips. The device or a system that includes the device can also include various additional circuitry components. Such components include, e.g., amplifiers, discriminators, converters and clocks. Any one or more of the components of the device and system can be included on one or more chips of the system.

The excitation source periodically interrogates the sensor with light. Light generated by the sensor in response to interrogation by the excitation source is detected by the detectors. The signals generated by the detector in response to the light received from the sensor are then communicated using wireless transmission to a remote device (e.g., PDA, computer (station, personal computer, laptop computer, or customized microprocessor), which analyzes the signals, optionally stores the signals, and optionally provides a visual display to the user. Where a visual display is provided, the display can correspond to a property of the analyte detected by the sensor including, e.g., the concentration of the analyte, the time at which the sample was obtained, trends associated with the data obtained, and combinations thereof.

The device can be maintained in position in relation to an implanted sensor with any suitable fixing means including, e.g., mechanical means (e.g., Velcro-type hook and loop fasteners) and adhesive means including, e.g., an adhesive tape, e.g., a BAND AID adhesive strip, which is adhered to the subject (e.g., the skin of the subject). The adhesive tape can include a clear plastic window and a sleeve and the device can be positioned in the sleeve of the tape. In preferred embodiments the adhesive is chosen to not cause allergic response in the patient.

The system can optionally include wave guides (e.g., optical fibers) to transmit and guide light from and to the excitation source and the detectors of the device. The light emitted by the excitation source, for example, can be transmitted to the sensor via a fiber optic. Likewise, light emitted from the sensor can be transmitted to one or more detectors via an optical fiber. The fiber optic can be in the form of an optical fiber (e.g., a multimode optical fiber or a single mode optical fiber), a bundle of optical fibers, or both. The fiber optic bundles can be random or patterned. Embodiments that include patterned fibers can be selected such that the patterned fibers are associated with a predetermined excitation source, detector and combinations thereof. In the case of fiber optic bundles, the bundle can be split into individual fibers or groups of fibers to carry the various different paths of light. In the case of optical fibers, a single optical fiber is positioned to transmit and guide a single path of light. Examples of useful wave guides include three dimensional fiber optic bundles and two dimensional wave guides in an optical circuit.

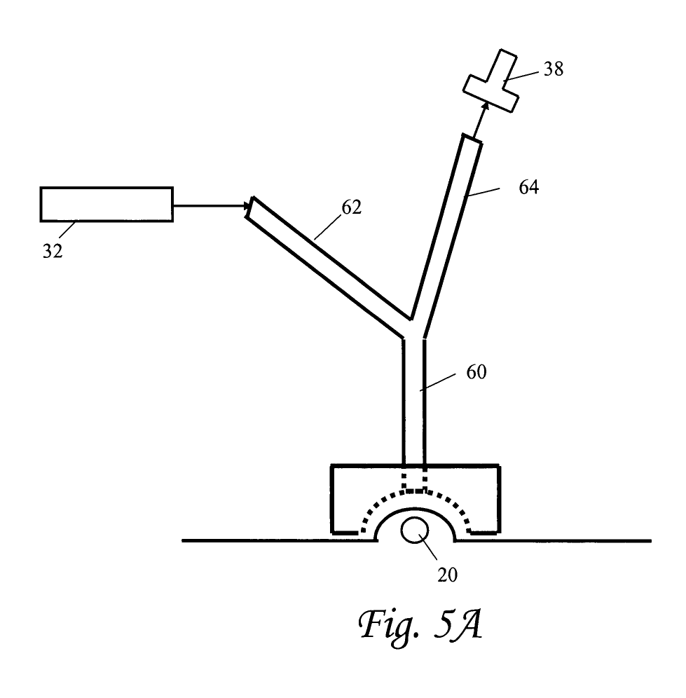

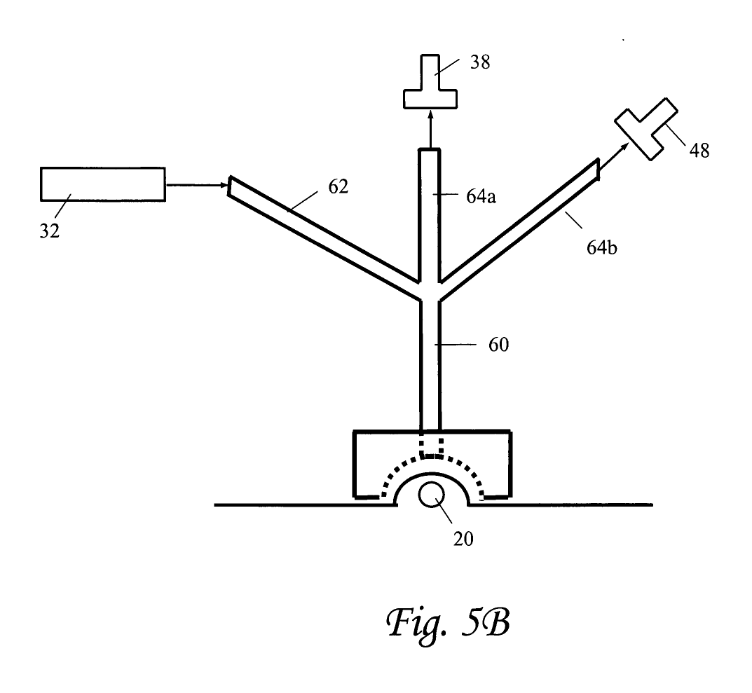

Referring to FIG. 5A, an embodiment of the device for detecting analyte includes an excitation source 32, a detector 38 and a fiber optic bundle 60. In operation, light emitted by the excitation source 32 travels through at least one fiber 62 of the fiber optic bundle 60 to the sensor 20 (including, e.g., the skin above the sensor). Fluorescence emitted by the sensor 20 travels through a second set of at least one fiber 64 of the fiber bundle 60 to the detector 38. The device optionally includes an optical filter to select the wavelength or range of wavelengths detected by the detector.

FIG. 5B illustrates an embodiment of the device for detecting analyte that includes an excitation source 32, two detectors 38, 48 and a fiber optic bundle 60. In operation, light emitted by the excitation source 32 travels through at least one fiber 62 of the fiber optic bundle to the sensor 20 or the skin above the sensor. Fluorescence emitted by the sensor 20 travels through the fiber optic bundle 60 to the detectors 38, 48. The fiber optic bundle 60 is split such that at least one each of the fibers 64a, 64b of the bundle 60 provide light to each detector 38, 48. The device optionally includes optical filters to select the wavelengths or range of wavelengths detected by each detector.

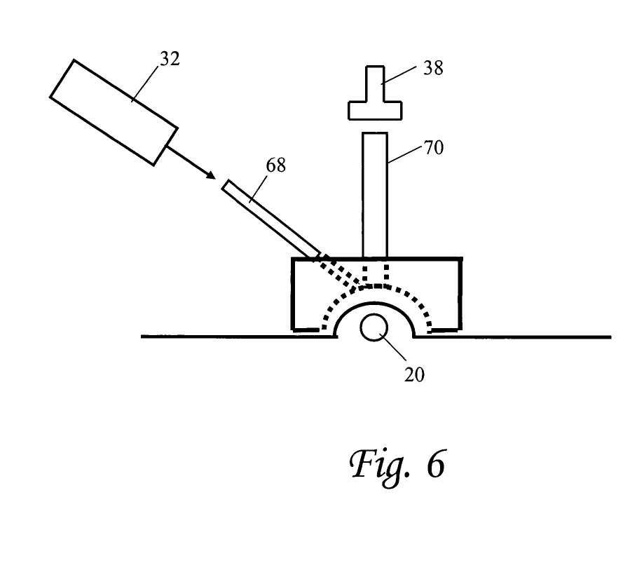

FIG. 6 illustrates an embodiment of the device for detecting analyte in which light emitted by the excitation source 32 travels through a first single mode optical fiber 68 to the sensor 20 and fluorescence emitted by the sensor 20 travels through an optical fiber 70 to a detector 38. The excitation radiation reaches the sensor 20 at an angle to the component collecting the fluorescence emitted by the sensor 20. Alternatively, the excitation radiation reaches the sensor 20 at a spaced apart relation (e.g., at an angle or laterally spaced relation) to the optical fiber collecting the fluorescence emitted by the sensor 20. The device optionally includes an optical filter to select the wavelength or range of wavelengths detected by the detector.

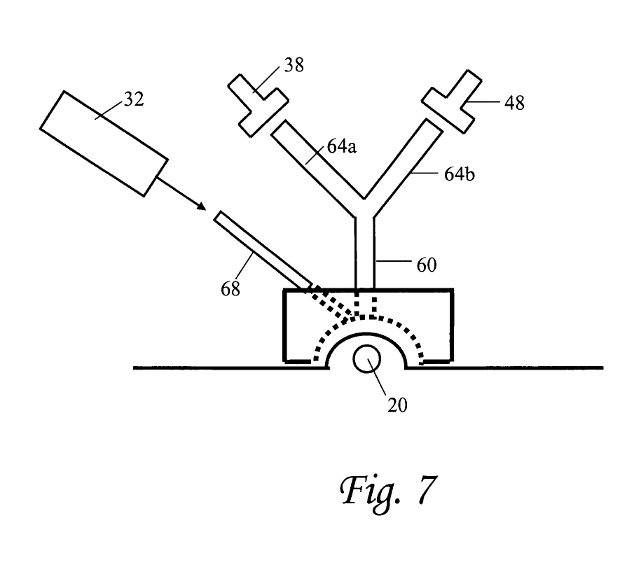

FIG. 7 illustrates an embodiment of the device for detecting analyte in which light emitted by the excitation source 32 travels through a first single mode optical fiber 68 to the sensor 20 and fluorescence emitted by the sensor 20 travels through a fiber optic bundle 60 to the two detectors 38, 48. The fiber optic bundle 60 is split such that at least one of the fibers 64a of the bundle provide light to the first detector 38 and at least one of the fibers 64b of the bundle provide light to the second detector 48. The device optionally includes optical filters to select the wavelengths or range of wavelengths detected by each detector.

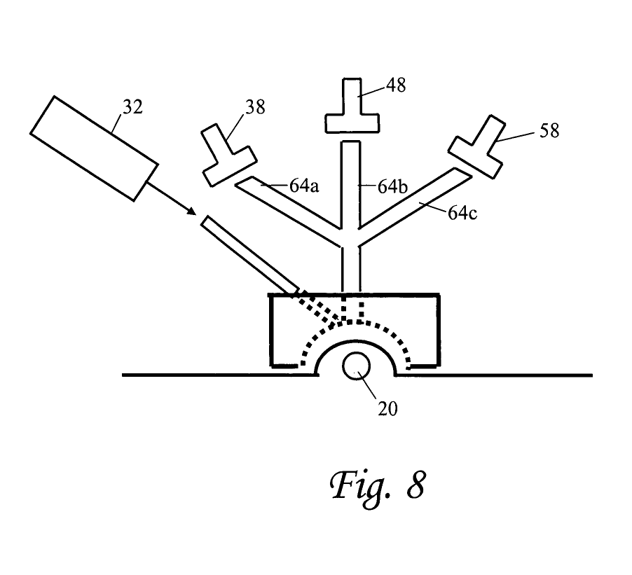

In FIG. 8 illustrates an embodiment of the device that includes a fiber optic bundle that is split into three separate units 64a, 64b, 64c, which carry fluorescence emitted by the sensor to the three detectors 38, 48, 58 of the device.

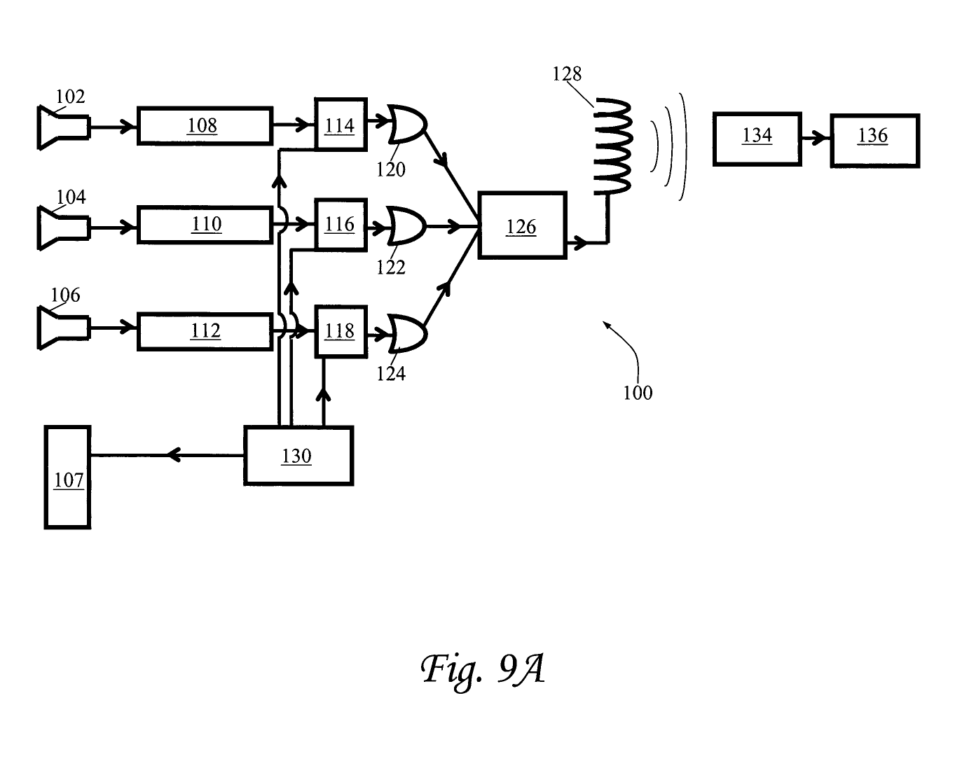

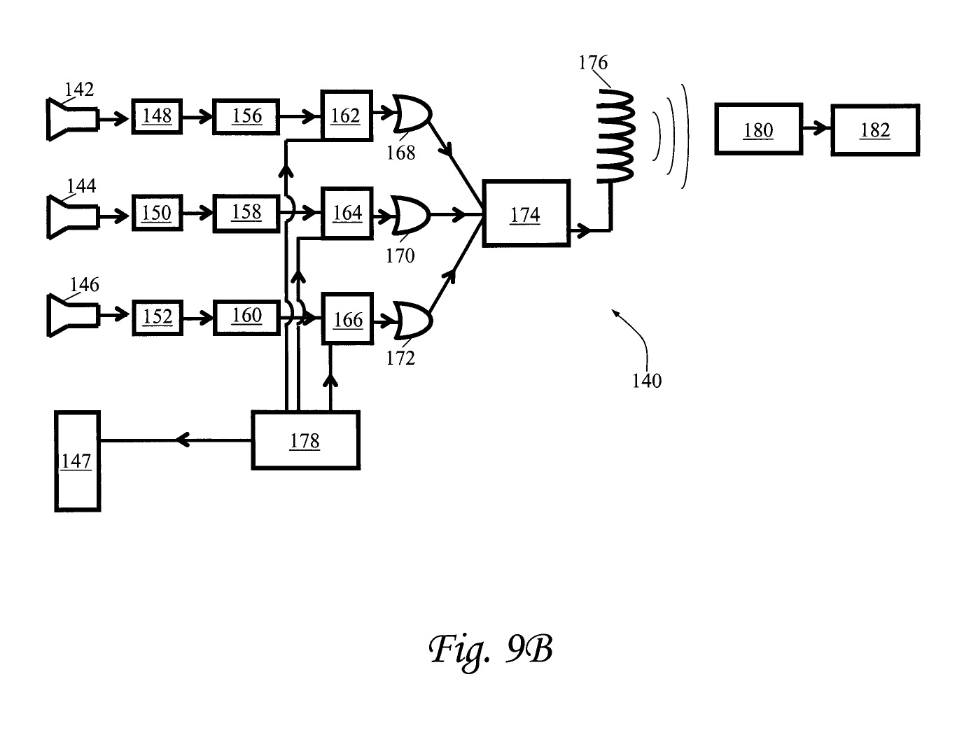

FIG. 9A is a block diagram showing a single-photon counting system 100. As shown in FIG. 9A, the components of the system 100 include three detectors 102, 104, 106, and an excitation source 107. The detectors 102, 104, 106 are coupled to amplifier/discriminators 108, 110, 112, which in turn are coupled to counters 114, 116, 118. The counters are coupled to conditional gates 120, 122, 124, which are coupled to a processor 126. The processor 126 may include a transmitter for transmitting signals via an antenna 128 to a remote site. The system 100 further includes a clock 130 for controlling operation of the excitation source 107 and the counters 114, 116, 118. The system 100 may optionally communicate with a remote site having a receiver 134 and an additional processor 136, as shown. The processors 126 and 136 may include embedded memory for data storage. While the system 100 shown in FIG. 9A operates using three detectors, amplifiers, counters, and gates, more or fewer numbers of each component could also be used.

During operation, a pulse from the clock 130 triggers the excitation source 107 to emit a pulse of light, which is positioned to excite fluorescence from the sensor. Fluorescence from the sensor and from the skin is collected by the detectors 102, 104, 106, each of which is adapted to detect an intensity at a particular wavelength (as described above). The detectors 102, 104, 106 collect photons and convert these photons to a current pulse, which is outputted to the amplifiers 108, 110, 112. The amplifier/discriminators 108, 110, 112 amplify the current pulses and convert all pulses above a predetermined threshold to a predetermined value and all pulses below this threshold to zero. During the emission of the light pulse by the excitation source 107, the clock also activates the counters 114, 116, 118 to count pulses received from the amplifier/discriminators 108, 110, 112. Counting is terminated by the conditional gates 120, 122, 124. In one embodiment, counting is terminated upon expiration of a certain amount of time (i.e., a specified number of clock pulses). According to another embodiment, counting is terminated after a predetermined count value is reached by one or two or all of the counters 114, 116, 118.

Next, in one embodiment, the count values are sent to the processor 126. In another embodiment, the time to reach a certain count value (e.g., a number of clock pulses) is sent to the processor 126. In one embodiment, the processor 126 totals the number of counts or clock pulses and converts these counts or clock pulses to an analyte concentration. In another embodiment, the processor totals the counts or clock pulses and then transmits these totals via antenna 128 to a receiver 134 for further processing by the additional processor 136. In this embodiment, the additional processor 136 then calculates the analyte concentration using an appropriate correlation between the signals acquired from the detectors 102, 104, 106 and the analyte concentration. In one embodiment, the analyte concentration is calculated using a formula that adjusts for background signal noise (e.g., skin fluorescence).

FIG. 9B is a block diagram showing an analog signal detection system 140. As shown in FIG. 9B, the components of the system 140 include three detectors 142, 144, 146, and an excitation source 147. The detectors 142, 144, 146 are coupled to amplifiers 148, 150, 152, which in turn are coupled to A/D converters 156, 158, 160. The A/D converters are coupled to initial processors 162, 164, 166, which, in some embodiments, are coupled to conditional gates 168, 170, 172. The conditional gates are coupled to a second processor 174. In another embodiment, the initial processors 162, 164, 166, are coupled directly to the second processor 174. In yet another embodiment, the system 140 does not include the initial processors 162, 164, 166. In this embodiment, the A/D converters are coupled directly to the second processor 174. The processors shown in FIG. 9B may include embedded memory for data storage. The second processor 174 may include a transmitter for transmitting signals via an antenna 176 to a remote source. The system 140 further includes a clock 178 for controlling operation of the excitation source 147 and the initial processors 162, 164, 166. The system 140 may optionally communicate with a remote site having a receiver 180 and an additional processor 182, as shown. While the system 140 shown in FIG. 9B operates using three detectors, amplifiers, A/D converters, initial processors, and gates, more or fewer numbers of each component could also be used.

During operation, a pulse from the clock 178 triggers the excitation source 147 to emit a pulse of light, which is positioned to excite fluorescence from the sensor. Fluorescence from the sensor and from the skin is collected by the detectors 142, 144, 146, each of which is adapted to detect an intensity at a particular wavelength (as described above). The detectors 142, 144, 146 convert the light intensity at its corresponding wavelength to either an analog current or voltage, which is then amplified by the corresponding amplifiers 148, 150, 152. The amplified signals are then digitized by the corresponding A/D converters 156, 158, 160. These digitized signals are then sent to a corresponding initial processor 162, 164, 166. The initial processors control the corresponding A/D converters and the data sampling rate. The initial processors may also be used to sum or average digital signals received from each of the detectors 142, 144, 146 for a given light pulse. As shown in FIG. 9B, the initial processors 162, 164, 166 are in communication with the clock 178, which allows the initial processors to operate in sequence with the light pulse emitted by the excitation source 147. The initial processors 162, 164, 166 may also operate to sum or average the digital signals received from multiple light pulses.

In the embodiments including the conditional gates 168, 170, 172, the conditional gates may operate to allow the data acquired by the initial processors 162, 164, 166 to pass to the second processor 174. In one embodiment, the conditional gates 168, 170, 172 allow data to pass to the second processor 174 after expiration of a predetermined time period. According to another embodiment, data is passed to the second processor 174 after a predetermined value is achieved in one or more of the initial processors 162, 164, 166.

In the embodiment of the system 140 where the A/D converters 156, 158, 160 are coupled directly to the second processor 174, the second transmitter is coupled directly to the clock 178. The second processor 174 then operates to control data acquisition and to control the A/D converters 156, 158, 160. In this embodiment, the second processor 174 stores and operates upon signals collected from each of the detectors 142, 144, 146 for a given light pulse and for accumulation of light pulses. In this embodiment, data acquisition is terminated by the second processor 174 upon expiration of a predetermined time period, or after a predetermined value is reached with respect to one of the detectors 142, 144, 146.

Next, in one embodiment, the second processor 174 calculates an analyte concentration using an appropriate correlation between the signal acquired from the detectors 142, 144, 146 and the analyte concentration. In another embodiment, the second processor 174 only acquires the data, either directly from the A/D converters 156, 158, 160 or from the initial processors 162, 164, 166, then transmits the data via antenna 176 to a receiver 180 for further processing by the additional processor 182. In this embodiment, the additional processor 182 then calculates the analyte concentration using an appropriate correlation between the signals acquired from the detectors 142, 144, 146 and the analyte concentration. In one embodiment, the analyte concentration is calculated using a formula that adjusts for background signal noise, such as skin fluorescence.

The various embodiments of the device can be constructed in a variety of forms including, e.g., a hand held pen-like device, a hand held gun-like device, a personal digital assistant, a wrist mounted enclosure (e.g., similar to a watch), a belt-mounted enclosure, and a strip of film positioned on the skin, e.g., with an adhesive (e.g., BANDAID adhesive strip). The device, the processor, the display and combinations thereof may be worn on a person, e.g., a person's wrist, upper arm, belt, glasses, or clothing. The excitation source and the detector(s), for example, can be worn in close proximity to an implanted sensor in various configurations including, e.g., a wrist watch-type configuration positioned over a sensor, and a belt mounted enclosure (e.g., similar to a beeper) used in conjunction with a sensor implanted near an individual's hip or waist.

The system can optionally include a variety of other components including, e.g., a pump capable of providing fluid (e.g., interstitial fluid and blood) from an individual to the sensor.

The system can be used to detect and analyze analytes in fluids provided to the sensor in an ex vivo application or in an in vivo application. A sensor is contacted with any suitable liquid of interest including, e.g., body fluids (e.g., blood, urine, extracellular fluid and interstitial fluid), the excitation source emits radiation to excite the fluorophores of the sensor and the detector(s) detects the fluorescence emitted by the sensor. One method of contacting the sensor with a fluid of interest includes positioning the sensor in a needle and pulling or passing the fluid through a needle such that the fluid contacts and permeates the sensor. In another method, the sensor is implanted such that a fluid of the body contacts the sensor.

In another method, an ex vivo sensor is removably positioned in the system and the fluid sample is brought into contact with the sensor using any suitable mechanism including, e.g., a pump capable of drawing fluid from the individual.

The system can be used in various applications and in a variety of industries including, e.g., the medical industry, the food industry, and the consumer products industry.

Other embodiments are within the claims. The detection device, for example, can include a sensor attached to the skin. In such a configuration, a needle or catheter can be placed beneath the skin to draw fluid, e.g., interstitial fluid or blood, to the sensor. In a second embodiment, the device is independent of the skin and the fluid is placed either in direct contact with the sensor or in a well from which the fluid is provided (e.g., by pumping, pushing, or drawing) to the sensor. Such devices can further include processors, pumps, and combinations thereof, that deliver medicament from a reservoir. The amount of medicament delivered can be determined based on a property of the analyte calculated by the processor. Alternatively, or in addition, the processor can communicate instructions to a pump via telemetry. Such devices can also further include physically independent processors that communicate with the device via telemetry.

The system and devices described herein are described with reference to a class of fluorescent sensor chemistries whose emission spectra change with analyte binding. Such chemistries enable simultaneous measurement at multiple wavelengths. Another suitable class of sensor chemistries exhibits a change in excitation spectra when analyte binding occurs. Useful examples of both of these classes of sensor chemistries are described in Haugland, Handbook of Fluorescent Probes and Research Products. Both Indo-1 and Fura-2, for example, are fluorescent chemistries that enable measurement of calcium ions. When excited at 338 nm Indo-1 fluorescence emission is dependent on calcium ions. The fluorescence emission of Fura-2 measured at 510 nm is preferentially excitable at different wavelengths depending upon the concentration of calcium ions. The various configurations described herein can be modified to accommodate sensor chemistries that exhibit a change in excitation, rather than emission, when analyte binding occurs. Such modifications include the use of multiple excitation sources in place of or in addition to the multiple detectors described above.

In other embodiments, wavelength separation can be effected with components other than or in addition to wavelength selecting filters including, e.g., prisms, diffraction gratings, and various combinations thereof. Prisms and diffraction gratings cause different regions of a spectrum to become spatially displaced and the spaced regions can then be detected at two or more detectors.

What is claimed is:

1. A system for detecting an analyte, the system comprising:
a sensor adapted to detect the analyte, the sensor comprising a polymer matrix, a fluorophore and a membrane surrounding the matrix;
an excitation source to excite the fluorophore;
a first detector adapted to detect light of a first wavelength emitted by the sensor;
a second detector adapted to detect light of a second wavelength emitted by the sensor; and
a processor for processing signals from the first and second detectors corresponding to light detected at the first and second detectors.

2. The system of claim 1, wherein the excitation source is adapted to transcutaneously excite the fluorophore.

3. The system of claim 1, wherein the detector is adapted to transcutaneously detect light emitted by the sensor.

4. The system of claim 1, further comprising a telemetry system for transmitting a signal corresponding to light detected at the first and second detectors to a remote location.

5. The system of claim 1, further comprising a first filter to filter light received by the first detector.

6. The system of claim 5, further comprising a second filter to filter light received by the second detector.

7. The system of claim 1, further comprising a first dichromatic mirror positioned to reflect light emitted by the excitation source and to transmit light emitted by the sensor.

8. The system of claim 7, further comprising a second dichromatic mirror positioned to reflect a first wavelength of light emitted by the sensor and to transmit a second wavelength of light emitted by the sensor.

9. The system of claim 1, further comprising a fiber optic operatively connected to the excitation source.

10. The system of claim 9, wherein the fiber optic comprises a single mode optical fiber.

11. The system of claim 1, further comprising a fiber optic operatively coupled to at least one of the detectors and adapted to transmit light from the sensor to the at least one detector.

12. The system of claim 1, further comprising a pump adapted to receive an instruction from the processor and to deliver an amount of a medicament, in response to the instruction.

13. The system of claim 1, wherein the fluorophores are mobile within the matrix.

14. The system of claim 1, wherein the processor is adapted to store a value corresponding to a property of the detected analyte as a function of time.

15. The system of claim 1, wherein the processor is adapted to transfer a value corresponding to a property of the detected analyte to a remote device.

16. The system of claim 1, wherein the processor is adapted to relate signals corresponding to the light detected by the detectors to a property of the analyte.

17. The system of claim 16, wherein the property comprises the concentration of the analyte.

18. The system of claim 1, wherein the processor is adapted to provide instructions regarding an activity related to a property of the detected analyte.

19. The system of claim 1, wherein the processor is adapted to provide instructions related to a property of the detected analyte to at least one of a mammal and a device.

20. The system of claim 19, wherein the instructions comprise instructions to administer least one of insulin, glucose or a combination thereof.

21. The system of claim 1, wherein the processor is adapted to provide an alarm when a predetermined condition related to a property of the analyte is met.

22. The system of claim 1, wherein the sensor detects analyte selected from the group consisting of carbohydrates, glycoproteins, glycopeptides, enzymes, glycolipids, hormones, lipoproteins, antibodies, antigens, haptens, steroids, theophylline, creatinine, drugs, polynucleotides, pesticides, and combinations thereof.

23. The system of claim 1, wherein the sensor detects glucose.

24. The system of claim 1, wherein at least one of the excitation source, the first detector and the second detector are located on at least one semiconductor wafer.

25. The system of claim 1, wherein the detectors are adapted to simultaneously detect light received from the sensor.

26. The system of claim 1, further comprising a means for pulsing the light emitted by the excitation source.

27. The system of claim 26, further comprising a means for phase locking the counting of signals at the detectors with the pulse emitted by the light pulsing means.

28. The system of claim 1, further comprising a pump adapted to draw fluid from an individual for contact with the sensor.

29. The system of claim 28, wherein the fluid comprises interstitial fluid or blood.

30. A system for detecting an analyte, the system comprising:
a sensor adapted to detect the analyte, the sensor comprising a polymer matrix, fluorophores, and a membrane;
an excitation source to excite a fluorophore of the sensor;
a first detector adapted to detect light of a first wavelength emitted by the sensor;
a second detector adapted to detect light of a second wavelength emitted by the sensor;
a third detector adapted to detect light of a third wavelength; and
a processor for processing signals corresponding to light detected by the detectors and for determining a property of the analyte.

31. The system of claim 30, wherein the excitation source is adapted to transcutaneously excite a fluorophore in the sensor.

32. The system of claim 30, wherein the detectors are adapted to transcutaneously detect light emitted by the sensor.

33. The system of claim 30 further comprising a transmitter for transmitting signals corresponding to light detected by the detectors to a remote location.

34. The system of claim 30 further comprising at least one filter to filter light received by at least one of the detectors.

35. The system of claim 30 further comprising a dichromatic mirror positioned to reflect light emitted by the excitation source and to transmit light emitted by the sensor.

36. The system of claim 35, further comprising a second dichromatic mirror positioned to reflect a first portion of the light transmitted through the first dichromatic mirror and to transmit a second portion of the light transmitted through the first dichromatic mirror.

37. The system of claim 36, further comprising a third dichromatic mirror positioned to reflect a first portion of the light transmitted through the second dichromatic mirror and to transmit a second portion of the light transmitted through the second dichromatic mirror.

38. The system of claim 30, further comprising a fiber optic operatively coupled to the excitation source.

39. The system of claim 30, further comprising a fiber optic operatively coupled to at least one of the detector to transmit light from the sensor to the at least one detector.

40. The system of claim 30, wherein the fiber optic comprises a bundle of optical fibers, a first portion of the fibers being operatively connected to the first detector, a second portion of the fibers being operatively connected to the second detector, and a third portion of the fibers being operatively connected to the third detector.

41. The system of claim 30, wherein the third detector is adapted to detect light emitted by skin when the skin is excited by the excitation source.

42. The system of claim 40, wherein the processor is programmed with code to correct for the light emitted and scattered by the skin.

43. The system of claim 30, wherein the processor is programmed with code to:
receive data corresponding to a first I(λ₁), a second I(λ₂), and a third I(λ_{B}) intensity measured at a first (λ₁), a second (λ₂), and a third (λ_{B}) wavelength, the third wavelength (λ_{B}) being selected such that the intensity detected at the third wavelength (λ_{B}) consists of background intensity;
correct the intensity at the first wavelength I(λ₁) based on the third fluorescence intensity I(λ_{B}) and a first predetermined correction function B(λ₁); and
correct the intensity at the second wavelength I(λ₂) based on the third intensity I(λ_{B}) and a second predetermined correction function B(λ₂).

44. The system of claim 43, wherein the processor further comprises code to calculate a ratio of the corrected intensity at the first wavelength (λ₁) to the corrected intensity at the second wavelength (λ₂).

45. The system of claim 43, wherein the processor further comprises code to determine a property of the analyte.

46. The system of claim 45, wherein the property is concentration.

47. The system of claim 30, wherein the processor is programmed with code to:
receive data corresponding to a first I(λ₁), a second I(λ₂), and a third I(λ_{B}) intensity at a first λ₁, a second λ₂, and a third λ₃ wavelength, respectively;
correct the intensity at the first wavelength I(λ₁) based on the intensity at the third wavelength I(λ₃) and a first set of three predetermined correction functions D(λ₁), A(λ₁), B(λ₁); and
correct the intensity at the second wavelength I(λ₂) based on the intensity at the third wavelength I(λ₃) and a second set of three predetermined correction functions D(λ₂), A(λ₂), B(λ₂).

48. A method of determining the concentration of an analyte using the system of claim 1, the method comprising
exciting a fluorophore located in the sensor,
detecting light of a first wavelength emitted by the sensor;
detecting light of a second wavelength emitted by the sensor, and
determining the concentration of the analyte based upon a corrected intensity of the light of the first wavelength and a corrected intensity of the light of the second wavelength.

49. The method of claim 48, further comprising determining the ratio of the intensity of the light emitted by the sensor at the first wavelength to the intensity of the light emitted by the sensor at the second wavelength.

50. The method of claim 48 further comprising determining the excited state fluorescence lifetime of the light emitted by the sensor at at least one of the first wavelength and the second wavelength.

51. The method of claim 48, wherein the sensor is implanted and the exciting comprises transcutaneously exciting fluorophores of the implanted sensor.

52. The method of claim 48, wherein the sensor is implanted and the detecting comprises transcutaneously detecting fluorophores of the implanted sensor.

53. The method of claim 48 further comprising transmitting signals corresponding to the detected light to a remote location.

54. The method of claim 48 further comprising detecting light of a third wavelength.

55. The method of claim 48 further comprising
drawing fluid comprising the analyte from an individual; and
contacting the sensor with the fluid.

56. A device comprising a detector-emitter array for detecting an analyte, the detector-emitter array comprising:
an excitation source adapted to excite a fluorophore of a sensor comprising fluorophores;
a first detector adapted to detect fluorescence light of a first wavelength emitted by the sensor;
a second detector adapted to detect fluorescence light of a second wavelength emitted by the sensor; and
a third detector adapted to detect light of a third wavelength.

57. The device of claim 56, further comprising a transmitter for transmitting a signal corresponding to light detected by the detectors to a remote location.

58. The device of claim 56, wherein the excitation source is adapted to transcutaneously excite a fluorophore of the sensor.

59. The device of claim 56, wherein the detector is adapted to transcutaneously detect light emitted by the sensor.

60. The device of claim 56, further comprising a processor for processing signals generated by the detectors.

61. The device of claim 60, wherein the excitation source is positioned to provide excitation radiation to a first area of the sensor and the detectors are positioned to detect light emitted from the sensor at a second area of the sensor, the first area being in a spaced apart relation to the second area.

62. The device of claim 61, further comprising an amplifier and an A/D converter for amplifying and digitizing the signal from the first detector.

63. The device of claim 62, further comprising a clock for controlling a duration of a pulse emitted by the excitation source and for controlling acquisition of first data, second data, and third data from the first, second, and third detectors.

64. The device of claim 63 further comprising a transmitter for transmitting the first data, the second data, and the third data to a remote location.

65. The device of claim 64 further comprising an additional processor for calculating a concentration of the analyte based on the first data, the second data, and the third data.

66. A system for detecting an analyte, the system comprising:
a sensor adapted to detect the analyte, the sensor comprising a polymer matrix, a membrane and fluorophores;
an excitation source to excite a fluorophore of the sensor;
a filter device for selecting from at least one of a first filter for filtering light of a first wavelength emitted by the sensor and a second filter for filtering light of a second wavelength emitted by the sensor;
a detector to detect light emitted by the sensor; and
a processor for processing signals corresponding to light detected by the detector.

67. The system of claim 66, wherein the filter device comprises a liquid crystal filter tunable to the first wavelength and the second wavelength.

68. A method of detecting fluorescence emitted by a sensor using the system of claim 66, the method comprising:
exciting fluorophores of the sensor;
detecting light of a first wavelength emitted by the sensor; and
subsequently detecting light of a second wavelength emitted by the sensor.

69. The method of claim 68, wherein the exciting comprises transcutaneously exciting fluorophores of the sensor.

70. The method of claim 68, wherein the detecting comprises transcutaneously detecting light emitted by the sensor.

71. A method of correcting a measured fluorescence intensity, the method comprising:
exciting a fluorophore of a sensor comprising fluorophores;
measuring a first I(λ₁), a second I(λ₂), and a third I(λ_{B}) intensity at a first a second λ₂, and a third λ₃ wavelength, respectively;
correcting the intensity at the first wavelength I(λ₁) based on the intensity at the third wavelength I(λ₃) and a first set of three predetermined correction functions D(λ₁), A(λ₁), B(λ₁); and
correcting the intensity at the second wavelength I(λ₂) based on the intensity at the third wavelength I(λ₃) and a second set of three predetermined correction functions D(λ₂), A(λ₂), B(λ₂).

72. The method of claim 71, further comprising
determining the fraction f₁ of the intensity due to emission by a first set of fluorescently labeled molecules D; and
determining the fraction f₂ of the intensity due to emission by a second set of fluorescently labeled molecules A.

73. The method of claim 72, wherein the determining is based on a predetermined first I_{D}(λ₁), I_{A}(λ₁), I_{B}(λ₁), second I_{D}(λ₂), I_{A}(λ₂), I_{B}(λ₂), and third I_{D}(λ₃), I_{A}(λ₃), I_{B}(λ₃) intensity of the first set of molecules D, the second set of molecules A, and the background B at each of the first (λ₁), the second (λ₂), and the third (λ₃) wavelengths, and the first I(λ₁), second I(λ₂), and third I(λ₃) intensities normalized to the intensity at the third wavelength λ₃.

74. The method of claim 71, wherein the determining is based on a first D(λ₁), A(λ₁), B(λ₁), second D(λ₂), A(λ₂), B(λ₂), and third D(λ₃), A(λ₃), B(λ₃) predetermined correction coefficient related to the first set of molecules D, the second set of molecules A, and the background B at each of the first (λ₁), the second (λ₂), and the third (λ₃) wavelengths.

75. The method of claim 71, wherein the determining comprises using the following equations:
$${f}_{1} = - \frac{- A ( {λ}_{3} ) * B ( {λ}_{2} ) + A ( {λ}_{2} ) B ( {λ}_{3} ) + A ( {λ}_{3} ) * I ( {λ}_{2} ) - B ( {λ}_{3} ) * I ( {λ}_{2} ) - A ( {λ}_{2} ) * I ( {λ}_{3} ) + B ( {λ}_{2} ) * I ( {λ}_{3} )}{A ( {λ}_{3} ) * B ( {λ}_{2} ) - A ( {λ}_{2} ) B ( {λ}_{3} ) - A ( {λ}_{3} ) * D ( {λ}_{2} ) + B ( {λ}_{3} ) * D ( {λ}_{2} ) + A ( {λ}_{2} ) * D ( {λ}_{3} ) - B ( {λ}_{2} ) * D ( {λ}_{3} )}$$
$${f}_{2} = - \frac{B ( {λ}_{3} ) * D ( {λ}_{2} ) - B ( {λ}_{2} ) D ( {λ}_{3} ) - B ( {λ}_{3} ) * I ( {λ}_{2} ) + D ( {λ}_{3} ) * I ( {λ}_{2} ) + B ( {λ}_{2} ) * I ( {λ}_{3} ) - D ( {λ}_{2} ) * I ( {λ}_{3} )}{- A ( {λ}_{3} ) * B ( {λ}_{2} ) + A ( {λ}_{2} ) B ( {λ}_{3} ) + A ( {λ}_{3} ) * D ( {λ}_{2} ) - B ( {λ}_{3} ) * D ( {λ}_{2} ) - A ( {λ}_{2} ) * D ( {λ}_{3} ) + B ( {λ}_{2} ) * D ( {λ}_{3} )}$$

76. A method of determining a property of an analyte in a fluid, the method comprising the method of claim 74 and further comprising calculating the ratio of the fluorescence intensity of the first set of molecules D to the fluorescence intensity of the second set of molecules A by dividing f₁ by f₂.

77. A method of determining the ratio of the fluorescence intensity of an energy donor D to the fluorescence intensity of an energy acceptor A of a sensor, the method comprising the method of claim 71 wherein calculating the ratio of the donor fluorescence intensity to the acceptor fluorescence intensity is based on a first D(λ₁), A(λ₁), B(λ₁), second D(λ₂), A(λ₂), B(λ₂), and third D(λ₃), A(λ₃), B(λ₃) predetermined fluorescence coefficient of the donor D, the acceptor A, and the background B at each of first (λ₁), the second (λ₂), and the third (λ₃) wavelengths and the first I(λ₁), second I(λ₂), and third I(λ₃) intensities normalized to the third I(λ₃) intensity.

78. The method of claim 77, wherein D is the donor fluorescence intensity due to direct excitation and A is the acceptor fluorescence intensity due to energy transfer.

79. A method of correcting for fluorescence intensity associated with a background component, the method comprising:
exciting a sensor comprising fluorophores;
measuring a first I(λ₁), a second I(λ₂), and a third intensity I(λ_{B}) corresponding to emission of the sensor at a first (λ₁), second (λ₂), and third wavelength (λ_{B}), the third wavelength (λ_{B}) being selected such that the intensity detected at the third wavelength (λ_{B}) consists of background signal;
correcting the intensity at the first wavelength I(λ₁) based on the third intensity I(λ_{B}) and a first predetermined correction function B(λ₁); and
correcting the fluorescence intensity at the second wavelength I(λ₂) based on the third fluorescence intensity I(λ_{B}) and a second predetermined correction function B(λ₂).

80. A method of determining a property of an analyte, the method comprising the method of claim 79 and further comprising calculating the ratio of the corrected intensity at the first wavelength (λ₁) to the corrected intensity at the second wavelength (λ₂).

81. The method of claim 80, wherein the property is the concentration of the analyte.

82. The method of claim 80, wherein the background signal is due to skin.

83. The method of claim 80, wherein the first wavelength is 600 nm, the second wavelength is 700 nm, and the third wavelength is 565 nm.
